(19) United States
(12) Patent Application Publication
(10) Pub. No.: US 2004/0101860 A1
(43) Pub. Date: May 27, 2004

(54) Predicting animal performance

(76) Inventors: Alison M. Jones, Eden Prairre, MN (US); W. Wade Robey, Excelsior, MN (US)

Correspondence Address: Mark S. Ellinger, Ph.D., Fish & Richardson P.C., P.A., Suite 3300, 60 South Sixth Street, Minneapolis, MN, 55402, US

(21) Appl. No.: 10/306,113

(22) Filed: Nov. 27, 2002

(51) Int. Cl. C12Q 1/68; G01N 33/554; G01N 33/569; G06F 19/00; G01N 33/48; G01N 33/50

(52) U.S. Cl. 435/6; 435/7.32; 702/20

(57) ABSTRACT

The invention provides for methods of characterizing animal performance based on the microbial profile of an animal. The invention also provides for methods of monitoring the effect of a feed or feed additive regimen on the microbial profile of one or more animals. The invention further provides for method of managing an animal growing or processing operation. Articles of manufacture are provided that can be used to evaluate the microbial profile of a biological sample, and systems and software are also provided for determining a microbial profile index from microbial profile information.

### TECHNICAL FIELD

This invention relates generally to animal performance, and more particularly to correlating the microbial profile of an animal with animal performance.

### BACKGROUND

Maximizing animal performance in food production animals is of considerable importance. The challenge in all forms of livestock production is to ensure that animal performance is optimum while costs are minimized. Since feed represents up to 70% of overall production costs in a swine or poultry operation, improving a performance characteristic such as efficiency of feed utilization can have a tremendous impact on producer profitability.

The genotype of an animal is one of the key factors affecting performance, as genotype determines an animal's maximum productive potential. While an animal's genotype is set at conception, a myriad of other factors interact to ultimately determine phenotypic expression. These factors include age, sex, physiological state (e.g., whether an animal is pregnant, or lactating), health status, feed intake, diet composition, management, and environment. Alterations in the microbial flora of the gastrointestinal tract (GIT) of an animal can impact animal performance. For example, in ruminants, a reduction in efficiency and numbers of ruminal microbes lowers the amount of available nutrients flowing from the rumen to the lower gut. When this occurs, animal performance is negatively affected. In monogastric animals, it is known that microbial populations tend to be homogeneous only within very discrete locations within the GIT, and that when considered as a whole, the GIT contains many different species of organisms, with only a fraction having been cultured and identified.

There are numerous approaches available for increasing animal performance such as manipulation of the diet density or supplementation of the diet with, for example, feed additives, administration of drugs to enhance growth, prevent disease or increase the efficiency of feed utilization, specialized housing arrangements, and selective breeding programs. Current approaches, however, do not reflect an understanding of the interrelationships among intestinal microbial populations (including those strains that colonize and those that remain free in the lumen), age, nutrition, health status, diet, and animal performance.

### SUMMARY

Animal performance can be affected, at least in part, by the microbial flora in, for example, the gastrointestinal tract (GIT) of the animal. The invention provides for methods of characterizing animal performance based on the microbial profile of an animal. In addition, the microbial flora in an animal is a dynamic community, and can be altered, for example, by changing an animal's diet. Therefore, the invention also provides methods of monitoring the effect of a feed or feed additive regimen on the microbial profile of one or more animals. The invention further provides methods of managing an animal growing or processing operation. Improved management can thereby optimize animal performance and the animal products produced therefrom. Articles of manufacture are provided that can be used to evaluate the microbial community of a biological sample and generate a microbial profile, and systems and software are also provided for determining a microbial profile index from microbial profiles.

In one aspect, the invention provides methods of characterizing animal performance, including determining a microbial profile in a biological sample from one or more animals; comparing the profile with a control microbial profile to determine a microbial profile index; and correlating the index with animal performance.

Characterizing animal performance can be performed on a plurality of animals. Animals for which animal performance can be characterized include avians such as poultry; porcines; ruminants; companion animals; aquatic animals; and humans. Representative animal performance characteristics include growth, weight gain, lean body mass, pigmentation, intake feed conversion, mortality, morbidity, rate of condemnation, happiness, health, and/or lack of sickness/illness.

The determining step can include microbial culturing, and colony identification, and can further include enumerating the colonies; histological analysis; immunological analysis; genetic fingerprinting; 16S rDNA genotyping; and/or cpn60 genotyping.

Generally, a control microbial profile is from a control biological sample taken from the animal prior to or subsequent to the taking of the biological sample, or a control microbial profile can be from a control biological sample taken from another animal. In addition, a control microbial profile can be from control biological samples taken from a plurality of animals. A baseline microbial profile can be from a database of microbial profiles.

Representative biological samples include gastrointestinal tract samples such as digesta, mucous and/or mucosal tissue, or feces; periodontal samples; bodily fluid such as blood, urine, saliva, sputum, or semen; soil samples; or aqueous samples. The biological samples can be pooled or examined individually.

A microbial profile determined using methods of the invention can be based on taxonomic and phylogenetic identification of microbes in the biological sample. Representative microbes include bacteria, protozoa, and fungi.

In another aspect, the invention provides methods of monitoring the effect of a feed or feed additive regimen on the microbial profile of one or more animals, including the steps of providing the feed or feed additive regimen to the animals; determining a post-regimen microbial profile of the animals; and comparing the post-regimen microbial profile with a pre-regimen microbial profile of the animals. Such a method can further comprise adjusting the feed or feed additive regimen based upon the comparison; and determining an adjusted-regimen microbial profile for the animals. Additionally, the method can even further comprise correlating the pre-regimen microbial profile, the post-regimen microbial profile, and/or the adjusted-regimen microbial profile with animal performance.

Representative feed additives include one or more components such as medicated feed additives, enzymes, probiotic microbials, direct-fed microbials, antimicrobials, prebiotics, phytochemicals, immunomodulators, antibodies, plant extracts, essential oils, organic acids, antioxidants, amino acids, oligosaccharides, oleoresins, herbs, spices, saponins, and sea plants.

In another aspect of the invention, there is provided an article of manufacture, including at least one cpn60 oligonucleotide, and instructions therein for using the cpn60 oligonucleotide(s) to characterize animal performance or to monitor the effects of a feed or feed additive on the microbial profile of the animal. Such cpn60 oligonucleotide(s) can be attached to a microarray.

In another aspect of the invention, there is provided a computer-readable storage medium having instructions stored thereon for causing a programmable processor to determine a microbial profile index for one or more animals based upon a microbial profile of the animals. The computer-readable storage medium can further comprise instructions for causing the programmable processor to correlate the microbial profile index with a performance characteristic of the animal. In addition, the computer-readable storage medium can further comprise instructions for causing the programmable processor to compare two profiles, wherein the comparison determines discreet differences between the profiles.

In another aspect of the invention, there is provided a system for determining a microbial profile index for one or more animals, the system including means for inputting subject microbial profile information; means for accessing control microbial profile information; and a processor for determining a microbial profile index. The system can further comprise means to obtain animal performance information based on the microbial profile index. The system can additionally comprise means to obtain instructions for improving the animal performance.

In yet another aspect of the invention, there is provided a method of managing an animal growing operation or an animal processing operation, including providing a microbial profile index for one or more animals; and communicating the index to a manager of the animal growing operation or the animal processing operation. The method can further include the step of correlating the index with animal performance; adjusting a feed or feed additive regimen; and/or modifying the animal processing operation.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the drawings and detailed description, and from the claims.

### DESCRIPTION OF DRAWINGS

FIG. 1 is a denaturing gradient gel electrophoresis (DGGE) analysis of 16S rRNA amplified from DNA extracted from replicate gastrointestinal tract (GIT) digesta samples at day 13 (A) and day 27 (B) from pigs fed a corn/soy diet or a corn/soy diet with sodium benzoate.

Like reference symbols in the various drawings indicate like elements.

### DETAILED DESCRIPTION

Animal performance can be affected, at least in part, by the microbial flora in, for example, the gastrointestinal tract (GIT) of the animal. Therefore, the invention provides for methods of characterizing animal performance based on the microbial profile of an animal. Animal performance can be described using one or more performance characteristics, including but not limited to, reproductive fitness/performance, body weight maintenance in the mature animal, rate of gain in growing animals, feed intake, feed conversion, mortality, morbidity, and carcass yield.

In addition, the microbial flora in an animal is a dynamic community, and can be altered, for example, by changing an animal's diet. Therefore, the invention also provides for methods of monitoring the effect of a feed or feed additive regimen on the microbial profile of one or more animals. Based on the effect of a feed or feed additive regimen on the microbial profile of one or more animals, the regimen can be adjusted to optimize the microbial profile of an animal.

The invention further provides methods of managing an animal growing or processing operation. Improved management can optimize animal performance and the animal products produced therefrom. Articles of manufacture also are provided that can be used to evaluate the microbial community (or a portion thereof) in a biological sample and generate a microbial profile, and systems and software are provided for determining a microbial profile index from microbial profiles.

### Microbial Profile

Microbial profiles are representations of individual strains, subspecies, species, and/or genera of microorganisms within a community of microorganisms. Generally, determining a microbial profile involves taxonomic and/or phylogenetic identification of the microbes in a community. A microbial profile can include quantitative information about one or more members of the community. Once one or more microorganisms have been identified in a microbial community, microbial profiles can be presented as, for example, lists of microorganisms, graphical or tabular representations of the presence and/or numbers of microorganisms, or any other appropriate representation of the diversity and/or population levels of the microorganisms in a community.

As used herein, “microbes” refer to bacteria, protozoa, fungi, and viruses. Microbial communities for which a microbial profile can be generated can include but are not limited to the following examples of prokaryotic genera: Staphylococcus, Pseudomonas, Escherichia, Bacillus, Salmonella, Bifidobacterium, Enterococcus, and Campylobacter; the following examples of protozoa genera: Acanthamoeba, Cryptosporidium, and Tetrahymena; the following examples of fungal genera: Aspergillus, Candida, and Saccharomyces; and the following viral genus: Coronaviridae.

A microbial profile in an animal can be determined using a number of methods. For example, the microbes in a sample can be cultured and colonies identified and/or enumerated. It has been estimated, however, that culturing typically recovers only about 0.1% of the microbial species in a sample (based on comparisons between direct microscopic counts and recovered colony-forming units). An improvement on culture-based methods is a community-level physiological profile. Such determinations can be accomplished by monitoring the capacity of a microbial community to utilize a suite of carbon sources with subsequent detection of the end product of this carbon metabolism by, for example, reduction of a tetrazolium dye. Profiling the physiology of a microbial community can yield qualitative (e.g., different patterns of reduced substrates) and semi-quantitative (e.g., spectrophotometric measurement of reduction) results. Biolog, Inc. (Hayward, Calif.) has commercialized a microtiter plate assay useful for determining the physiological profile of a complex microbial community. The BIOLOG method requires a standard inoculum density of metabolically active microorganisms, and assumes that all members of the community grow at the same rate so the utilization profile is not skewed by the metabolic capabilities of the fastest growers, and further assumes that the 95 substrates reflect the comprehensive substrate availability in the environment of interest.

Culture-independent methods to determine microbial profiles consist of extracting and analyzing microbial macromolecules from a sample. In general, useful target molecules are ones that, as a class, are found in all microorganisms, but are diverse in their structures, thereby reflecting the diversity of the microbes. Examples of target molecules include phospholipid fatty acids (PLFA), polypeptides, and nucleic acids. PLFA analysis is based on the universal presence of modified fatty acids in microbial membranes, and is useful as a taxonomic tool. PLFAs are easily extracted from samples, and separation of the various signature structures reveals the presence and abundance of classes of microbes. This method requires appropriate signature molecules, which often are not known or may not be available for the microbes of interest. In addition, the method requires that an organism's PLFA content does not change under different metabolic conditions. Another limitation to using PLFAs as target molecules is that widely divergent organisms may have the same signature set of PLFAs.

Other less direct measures can be made that can provide insight into changes that might be taking place in the microbial profile within the GIT of animals. For example, GIT tissues can be excised and histologically evaluated for the number, size, shape, mucosal-cell turnover and condition of the villi. The microscopic appearance of the villi can correlate with changes in the microbial ecology of the animal, as many of the resident organisms attach directly to the mucosa, and have been previously reported to be capable of causing damage and/or destruction of the absorptive surface. Examples include direct damage that occurs in regions of the GIT when animals are suffering from necrotic enteritis, non-specific enteritis, or other known GIT disease that are the result of pathogenic microbes.

Techniques such as immunohistochemical analysis also can be employed as indicative measures of pathogenic changes in the GIT profile for an animal. An increased presence of circulating leukocytic cytokines (lymphokines and monokines), as well as the presence of immunoglobulins (e.g., IgM, IgG, or IgA) either in systemic circulation or localized in GIT tissue at the site of a gastric antigenic insult can be examined and used to evaluate potentially deleterious changes in the microbial ecology in the animal.

Various nucleic acid-based assays can be employed to determine a microbial profile for an animal. For example, some nucleic acid-based population methods use denaturation and reannealing kinetics to derive an indirect estimate of the percent (%) guanine and cytosine nucleotides (G+C) content of the DNA in the sample. This method has been used to characterize the total bacterial community in the ileum and cecum of the GIT in poultry, and to examine how diet and other variables modulate the microbial communities in the GITs of animals (Apajalahti et al., 2001, Appl. Environ. Microbiol., 67:5656-67). The % G+C technique provides an overall view of the microbial community and is sensitive only to massive changes in the make-up of the community.

Genetic fingerprinting of a sample from an animal is another method that can be used to determine a microbial profile for an animal. Genetic fingerprinting utilizes random-sequence oligonucleotide primers that hybridize with sequence-specificity to random sequences throughout the genome. Amplification results in a multitude of products. The distribution of amplification products is referred to as a genetic fingerprint. Particular patterns can be associated with a community of microbes in the sample. Genetic fingerprinting, however, lacks the ability to conclusively identify specific microbial species.

Denaturing or temperature gradient gel electrophoresis (DGGE or TGGE) is another technique that can be used to determine a microbial profile for an animal. As amplification products are electrophoresed in gradients with increasing denaturant or temperature, the double-stranded molecule melts and its mobility is reduced. The melting behavior is determined by the nucleotide sequence, and unique sequences will resolve into individual bands. Thus, a D/TGGE gel yields a genetic fingerprint characteristic of the microbial community, and the relative intensity of each band reflects the abundance of the corresponding microorganism. An alternative format includes single-stranded conformation polymorphism (SSCP). SSCP relies on the same physical basis as % G+C renaturation methods, but reflects a significant improvement over such methods.

In addition, a microbial profile for an animal can be determined using terminal restriction fragment length polymorphism (TRFLP). Amplification products can be analyzed for the presence of known sequence motifs using restriction endonucleases that recognize and cleave double-stranded nucleic acids at these motifs. For example, the enzyme Hhal cuts at 5′-GCGC-3′ sites. Using a fluorescently-labeled primer to tag one end of the amplification product and Hhal to digest the products, resolution of this mixture by electrophoresis will yield a series of fluorescent bands whose lengths are determined by how far a 5′-GCGC-3′ motif lies from the terminal tag. TRFLP profiles can be generated using a variety of restriction enzymes, and can be correlated with changes in the population. For example, a TRFLP database for 16S rRNA sequences has been set up at Michigan State University to allow researchers to design experimental parameters (e.g., choice of enzyme and primer combinations). The principal advantages of TRFLP are its robustness and its low cost. Unlike D/TGGE, experimental conditions need not be stringently controlled since the profiles are size-based and thus can be generated by a variety of gel systems, including automated DNA sequencing machines. Alternative approaches include “amplified ribosomal DNA restriction analysis (AADRA)” in which the entire amplification product, rather than just the terminal fragment, is considered. AADRA, however, becomes unmanageable with communities containing many species.

Genotyping of 16S ribosomal DNA (rDNA) is another way to determine a microbial profile for an animal. 16S rDNA sequences are universal, composed both of highly conserved regions, which allows for the design of common amplification primers, and open reading frame (ORF) regions with sequence variation, which allows for phylogenetic differentiation. 16S ribosomal sequences are relatively abundant in the RNA form. In addition to amplification using oligonucleotide primers, genotyping of 16S rDNA can be performed using other methods including restriction fragment length polymorphism (RFLP) with Southern blotting.

Other targets for genotyping include genes encoding components of RNA polymerase, translation elongation factors, gyrase, and chaperoning. Such protein-encoding sequences may evolve more rapidly than those encoding structural RNAs. Thus, the sequences of protein-encoding sequences in closely related species may have diverged more in closely related species and may provide more discriminatory information. The choice of which target sequence to use depends on whether the sequences provide both broad coverage and discriminatory power. Ideally, the target should be present in all members of a given microbial community, be amplified from each member with equal efficiency using common primers, yet have distinct sequences. Multiple targets may in fact prove necessary for particular applications.

Chaperonin 60 (cpn60) nucleic acid sequences are particularly useful targets for genotyping and can be used to determine a microbial profile of an animal. Chaperonin proteins are molecular chaperones required for proper folding of polypeptides in vivo. cpn60 is found universally in prokaryotes and in the organelles of eukaryotes, and can be used as a species-specific target and/or probe for identification and classification of microorganisms. Sequence diversity within this protein-encoding gene appears greater between and within bacterial genera than for 16S rDNA sequences, thus making cpn60 a superior target sequence having more distinguishing power for microbial identification at the species level than 16S rDNA sequences.

PCR oligonucleotide primers that universally amplify a 552-558 base pair (bp) segment of cpn60 from numerous microorganisms have been generated (see, for example, U.S. Pat. Nos. 5,708,160 and 5,989,821), and the nucleotide sequence of this region of cpn60 has been evaluated as a tool for microbial analysis. The utility of the sequence diversity in cpn60 has been demonstrated, in part, by cross hybridization experiments using nylon membranes spotted with cpn60 amplification products from typed strains probed with labeled amplification product from unknown isolates. By manipulating stringency conditions, hybridization can be limited to targets having >75% identity (e.g., >80%, >85%, >90%, >95% identify) to the unknown isolate. This level of cross hybridization allows for clear differentiation of species within genera.

Nucleic acid hybridization is another method that can be used to determine a microbial profile for an animal. Probing amplification products with species-specific hybridization probes is one of the most powerful analytical tools available for profiling. The physical matrix for hybridization can be nylon membranes (e.g., macroarrays) or microarrays (e.g., microchips), incorporation of the hybridization probes into the amplification reaction (e.g., TaqMan or Molecular Beacon technology), solution-based methods (e.g., ORIGEN technology), or any one of numerous approaches devised for clinical diagnostics. Probes can be designed to preferentially hybridize to amplification products from individual species or to discriminate species phylogenetically.

A microbial profile also can be determined for an animal by cloning and sequencing microbial nucleic acids present in a biological sample from such an animal. Cloning of individual nucleic acids into Escherichia coli and sequencing each nucleic acid gives the highest density of information but requires the most effort. Although sequencing nucleic acids is automated, routine monitoring of changes in the microbial profile of an animal by cloning and sequencing nucleic acids from the microorganisms still requires considerable time and effort.

Many of the nucleic acid-based methods described above rely upon amplification of nucleic acids. Amplification methods such as the polymerase chain reaction (PCR) are particularly powerful methods to increase the amount of a particular nucleic acid sequence. Amplification reactions usually use two oligonucleotide primers. An oligonucleotide primer typically contains 12-20 or more nucleotides. The exact length of an oligonucleotide primer will depend on many factors, including the temperature of the reaction, and the reaction buffer. The oligonucleotide primers to be used in an amplification reaction typically have similar melting temperatures. Conditions under which extension can occur include the presence of nucleoside triphosphates, a polymerase enzyme such as DNA polymerase, and a suitable temperature and pH. An oligonucleotide primer is usually single-stranded, but can be double-stranded. If double-stranded, a primer is first denatured before template nucleic acid is introduced. A primer must be long enough to anneal with sequence-specificity and to initiate extension.

The template nucleic acid to be amplified does not need to be present in a pure form; it may be a minor fraction of a complex mixture, such as contained in a biological sample from an animal. Where the template nucleic acid sequence of the sample is double-stranded, it is necessary to separate the strands of the template nucleic acid. Strand separation can be effected either as a separate step or simultaneously with annealing of the primers and extension therefrom. Denaturing template nucleic acid can be accomplished using physical, chemical, or enzymatic means. One method of denaturing nucleic acid strands involves heating the nucleic acid until the two strands are separated. Typically, denaturation involves temperatures ranging from about 80° to 105° C., for times ranging from about 1 to 10 minutes.

Amplification is an enzymatic reaction that increases the number of nucleic acid molecules. Typically, a first oligonucleotide primer is complementary to one strand of the template nucleic acid and a second oligonucleotide primer is complementary to the other strand. Annealing the oligonucleotide primers to denatured template nucleic acid followed by extension with a polymerase enzyme, such as Taq Polymerase, in the presence of nucleotides results in newly synthesized nucleic acid molecules containing the template nucleic acid sequence. Because these newly synthesized sequences also can act as templates, repeated cycles of denaturing, primer annealing, and extension typically results in exponential production of the template nucleic acid. The nucleic acid molecules produced following amplification are usually discrete nucleic acid duplexes with termini corresponding to the ends of the specific oligonucleotide primers employed.

Oligonucleotide probes are often used in hybridization reactions to detect nucleic acids. Oligonucleotide probes must be sufficient in length for sequence-specific hybridization to occur. Oligonucleotide probes are generally 15 to 30 nucleotides in length or longer. In addition, oligonucleotide probes can be designed to hybridize to targets that contain a polymorphism or mutation, thereby allowing differential detection of microorganisms based on either absolute hybridization of one or more oligonucleotide probes corresponding to a particular microorganism or differential melting temperatures between, for example, one or more oligonucleotide probes and nucleic acid from the microorganisms to be distinguished.

### Animal Performance

Performance characterization is a vital component of animal production. Heretofore, producers have relied heavily on more traditional performance measures such as weight gain, feed conversion, mortality, morbidity, and carcass yield. It is disclosed herein that animal performance in economically important species such as poultry, bovine, ovine, and ruminants can be characterized on the basis of their microbial flora in, for example, their GIT, using the methods of the invention. Microbial profiles can be determined as described herein and can be used in the methods of the invention to characterize and/or predict animal performance. Animal performance is generally associated with the overall condition of the animal, but also can be described by one or more specific performance characteristics.

Animal growth is a performance characteristic that can be used to characterize animal performance. Generally, animal growth refers to an increase in body height, length, girth and/or weight of an animal. Growth velocity of an animal is curve-linear during the commercial growing period. Live weight is an important and commonly measured performance characteristic and, if recorded at more than one interval, can be used to plot a growth curve for the animal.

Similarly, lean body mass is a performance characteristic that can be used to characterize animal performance. Lean body mass can be evaluated in an animal at various time points during the grow-out cycle via a variety of invasive or non-invasive techniques including direct body-compositional analysis, electrical impedance, and ultrasound.

Feed conversion is another performance characteristic that can be used to characterize animal performance. Feed conversion is defined as a ratio of the quantity of feed necessary per unit of gain, and is a calculated variable using body weight and feed intake data. Although it is desirable to measure feed conversion during various points in the animal's growth under commercial conditions, feed intake generally cannot be calculated until harvest or slaughter.

Pigmentation is a measure of nutrient utilization and also can be used to characterize animal performance. Pigmentation changes in the skin, shanks, and beak of poultry, for example, can be indicative of declining animal performance. Evaluating pigmentation in an animal can be performed at any time during the growing operation and is usually performed by visual inspection.

Mortality and/or morbidity is an important benchmark of animal performance. Mortality rates can be readily determined by evaluating the number of deaths in a population of animals. Morbidity can be evaluated by a decrease in, for example, body weight and/or feed conversion, and can be observed at any time during the growing operation or at a specific time such as just prior to slaughter.

Similarly, the rate of condemnation can be used to characterize animal performance. Rates of condemnation are generally determined in a processing plant and reflect field morbidity and contamination.

In addition to the food production animals discussed above, humans, human companion animals (e.g., cats, or dogs), and aquatic species can be evaluated for performance. Many of the performance characteristics described above (e.g., growth and weight gain) can be applied to such species. In addition, performance in humans and companion animals can be characterized based upon health and/or happiness. Health of an animal can be assessed by determining the number of sicknesses or illnesses experienced during a period of time. It can be appreciated that happiness is most readily measured in human subjects using, for example, a personality test such as one to evaluate a person's self-esteem. See, for example, Sapountzi-Krepia et al., 2001, J. Adv Nurs., 35(5):683-90; Neto, 2001, Phychol. Resp., 88(3P+1):817-24; and Diener, 2000, Am. Psychol., 55(1):34-43. Nevertheless, particularly with companion animals, happiness can be evaluated by the extent to which they express themselves (e.g., barking, purring, tail wagging, etc.).

### Determining A Microbial Profile Index

Once a microbial profile has been determined for an animal or population of animals, it can be compared to a control microbial profile. A control microbial profile is determined using a control biological sample in the same manner in which a microbial profile is determined using a biological sample from a subject animal (i.e., a “subject biological sample”). In one embodiment, a control biological sample can be obtained from the same subject animal, and can be taken prior to or subsequent to the taking of the subject biological sample. In another embodiment, a control microbial profile can be determined for a control biological sample taken from an animal other than that from which the subject biological sample was obtained. In yet another embodiment, control biological samples can be obtained from a plurality of animals. Such control biological samples from a plurality of animals can be analyzed individually and evaluated for their microbial profiles, or such control samples can be pooled and analyzed collectively for a microbial profile.

In one embodiment, a microbial profile determined for a subject animal is compared to one or more control microbial profiles in a database of microbial profiles. A database of microbial profiles can include microbial profiles for only one species of animal, or a database can include microbial profiles for multiple species. Distribution of such a database can be by physical media such as CD-ROM, floppy disks, or tapes, or by electronic downloads via dial-up or network connections.

The difference between a subject microbial profile and a control microbial profile is termed the “microbial profile index.” The microbial profile index can be a series of numerical values corresponding to the presence, absence, or the difference in number of each microbial species in a subject biological sample compared to a control biological sample. Alternatively, the microbial profile index can be a single value representing cumulative differences between the microbial profile determined for a subject biological sample and a control biological sample. For example, a subject microbial profile and a control microbial profile each can be represented by a line graph, where each point on the line graph corresponds to the number of organisms present for a particular microbial species. In this case, for example, a microbial profile index can be determined by subtracting the area under the curve corresponding to the subject microbial profile from the area under the curve corresponding to the control microbial profile, and assigning a numerical value to the difference in areas. In other embodiments, a value can be assigned to each genus of microbes, and can be used to represent, for example, the diversity in species within each genus and the number of microorganisms present from each species.

It can be appreciated by those of ordinary skill in the art that numerous methods are available for calculating a microbial profile index, and the particular method used to determine a microbial profile index may be dependent upon such factors as the complexity of the microbial community or the end-user to which the microbial profile index is reported.

### Method of Characterizing an Animal's Performance

The invention provides methods that can be used to characterize animal performance. Methods of characterizing animal performance include evaluating the microbial profile of one or more subject animals using a biological sample from the subject animal(s), and comparing the microbial profile obtained for a subject animal with a control microbial profile to determine a microbial profile index. The microbial profile index then can be correlated with animal performance.

Using the methods of the invention, animal performance can be characterized in individual animals or in a plurality of animals (e.g., a population, or a herd). As discussed above, the methods of the invention can be used to characterize animal performance in commercially valuable animals such as avian animals (e.g., poultry), porcine animals, or ruminant animals. Animal performance also can be characterized in aquatic and companion animals (e.g., pets), as well as in humans.

As used herein, “biological sample” refers to any sample obtained directly from a subject animal or control animal, or any sample that has been in contact with such an animal, and from which microbes or microbial nucleic acid can be isolated. Representative biological samples that can be obtained directly from an animal include, but are not limited to, samples obtained from the gastrointestinal tract (e.g., digesta, mucous and/or mucosal tissue, or feces), from the mouth or teeth (i.e., a periodontal sample), or from bodily fluid such as blood, urine, saliva, sputum, or semen. Such biological samples can be obtained from an animal using methods and techniques known in the art. See, for example, Diagnostic Molecular Microbiology: Principles and Applications (Persing et al. (eds), 1993, American Society for Microbiology, Washington D.C).

Additionally, a biological sample suitable for use in the methods of the invention can be a substance that one or more animals have contacted. For example, an aqueous sample from a water bath, a chill tank, a scald tank, or other aqueous environments with which a subject or control animal has been in contact, can be used in the methods of the invention to evaluate a microbial profile. A soil sample that one or more subject or control animals have contacted, or on which an animal has deposited fecal or other biological material, also can be used in the methods of the invention. Nucleic acids can be isolated from such biological samples using methods and techniques known in the art. See, for example, Diagnostic Molecular Microbiology: Principles and Applications (Persing et al. (eds), 1993, American Society for Microbiology, Washington D.C).

The microbial community in one or more animals can be evaluated as described herein, and a microbial profile generated. The microbial profile then can be compared to a control microbial profile. A comparison between the microbial profile for the subject animal and an appropriate control microbial profile yields a microbial profile index, as described above. The microbial profile index then can be used to characterize animal performance in the subject animal. The correlation between a particular microbial profile index and animal performance can be based upon a dataset of microbial profiles for animals exhibiting varying degrees of performance (e.g., high performers to low performers). Animal performance can be described by a single performance characteristic (e.g., growth), but is often described by a number of performance characteristics.

### Methods of Monitoring the Effect of a Feed or Feed Additive Regimen on Microbial Profile

The invention further provides for methods of monitoring the effect of a feed or feed additive regimen on the microbial profile of one or more animals. Such a method includes providing a feed or feed additive regimen to one or more of the subject animals, determining a post-regimen microbial profile for one or more of the subject animals, and comparing the post-regimen microbial profile with a pre-regimen microbial profile. Generally, a post-regimen microbial profile refers to the microbial profile of one or more subject animals after a feed or feed additive has been introduced. Similarly, a pre-regimen microbial profile generally refers to the microbial profile of the subject animals prior to introducing a feed or feed additive regimen. As used herein, however, a pre-regimen microbial profile also can refer to the microbial profile of subject animals to which the feed or feed additive has been provided and then removed, measured after the subject animal has been allowed to return to normalcy.

If desirable, the feed or feed additive regimen provided to the subject animal(s) can be adjusted such that the microbial profile in the subject animal(s) is altered. An adjusted-regimen microbial profile then can be evaluated in the subject animal(s). The feed or feed additive regimen can be adjusted as many times as is necessary to obtain the desired microbial profile in the animal(s). Optionally, the pre-regimen microbial profile, the post-regimen microbial profile, and/or the adjusted-regimen microbial profile can be used to determine a microbial profile index (e.g., a pre-regimen microbial profile index, a post-regimen microbial profile index, or an adjusted-regimen microbial profile index, respectively), which then can be used to characterize performance in the subject animal(s).

As used herein, “feed additive” refers to any number of components that can be added to feed or to the diet of an animal. Feed additives include, but are not limited to, medication, enzymes, probiotic microbials, direct-fed microbials, antimicrobials, prebiotics, phytochemicals, immunomodulators, antibodies, plant extracts, essential oils, organic acids, antioxidants, amino acids, oligosaccharides, oleoresins, herbs, spices, saponins, and sea plants. Animal growers and producers routinely use such feed additives to try to optimize animal performance while keeping costs to a minimum. The methods of the invention allow growers and produces to continually monitor the effect of a feed or feed additive on animal performance in a single animal or in a population of animals.

### Methods of Managing Animal Growing Anchor Processing Operations

The invention also provides for methods of managing an animal growing operation or an animal processing operation. Such methods include communicating a microbial profile index determined for one or more animals to a manager of the animal growing operation or animal processing operation. As used herein with reference to a growing or processing operation, a “manager” refers to any individual(s) associated with managing the operation and making decisions concerning the animals in such an operation. As used herein, a “manager” can oversee the operations of a growing or processing facility, or a “manager” can be a caretaker, the person responsible for feeding the animals, or the person responsible for slaughtering or preparing the animals for slaughter. As used herein, representative “managers” include live production staff, veterinary staff, and nutritionists within an animal production operation.

In an embodiment of the invention, the microbial profile of an animal or of a population of animals can be determined on-site at an animal growing or processing facility. To determine microbial profiles on-site would require that the facility possess the equipment necessary to obtain a biological sample and evaluate the microbial profile therefrom. Depending upon the assay format used, the equipment necessary to evaluate microbial profile can be, for example, a thermocycler, or instruments to process and analyze microarrays.

In another embodiment, microbial profiles can be determined off-site from the growing or processing operation. A manager or employee of the facility can obtain a biological sample from one or more subject animals and provide the sample to an outside source (e.g., a diagnostic laboratory) to be evaluated for its microbial profile. Alternately, a representative from an outside source (e.g., an outside contractor or vendor) can obtain the biological sample from one or more subject animals and evaluate the microbial profiles of such samples.

Upon receipt of a microbial profile index, managers of an animal growing operation may wish to correlate the microbial profile index with animal performance. Based on the microbial profile index itself, or in combination with the animal performance characterized therefrom, managers of an animal growing operation can adjust a feed or feed additive regimen to improve animal performance or to maintain animal performance while lowering production costs. In addition, managers may wish to identify animals exhibiting low performance or a microbial profile index indicating such, and remove those animals from the population.

Similarly, managers of an animal processing operation may wish to modify the animal processing operation based upon the microbial profile index or the animal performance characterized therefrom. For example, a manager of an animal processing operation can change the order that populations of animals are processed or remove a population of animals from the processing operation if, for example, one population of animals has an elevated level of a microorganism that is pathogenic to humans (e.g., certain E. coli or Salmonella spp.).

### Articles of Manufacture

The invention also provides for articles of manufacture. Articles of manufacture of the invention can include at least one cpn60 oligonucleotide primer, as well as instructions for using the cpn60 oligonucleotide(s) to evaluate a microbial profile or to monitor the effects of a feed or feed additive on the microbial profile of an animal. An article of manufacture of the invention also can include instructions for obtaining a microbial profile index from a microbial profile, and further can include instructions for characterizing animal performance using the microbial profile index.

In one embodiment, the cpn60 oligonucleotide(s) are attached to a microarray (e.g., a GeneChip®, Affymetrix, Santa Clara, Calif.). In another embodiment, an article of manufacture can include one or more cpn60 oligonucleotide primers and one or more cpn60oligonucleotide probes. Such cpn60 primers and probes can be used, for example, in real-time amplification reactions to amplify and simultaneously detect the amplification product.

Suitable oligonucleotide primers are those that are complementary to highly conserved regions of cpn60 and that flank a variable region. Such cpn60 primers can be used to specifically amplify these variable regions, thereby providing a sequence with which to identify microorganisms. Representative cpn60 oligonucleotide primers can have the following sequences:

| | |
|---|---|
| 5′-GAIIIIGCIGGIGA(T/C)GGIACIACIAC-3′ | (SEQ ID NO:1) |
| and | |
| 5′-(T/C)(T/G)I(T/C)(T/G)ITCICC(A/G)AAICCIGGIGC(T/C)TT-3′. | (SEQ ID NO:2) |

Similar to cpn60 oligonucleotide primers, cpn60 oligonucleotide probes are generally complementary to cpn60 sequences. Cpn60 oligonucleotide probes can be designed to hybridize universally to cpn60 sequences, or can be designed for species-specific hybridization to the variable region of cpn60 sequences.

An article of manufacture of the invention can further include additional components for carrying out amplification reactions and/or reactions, for example, on a microarray. Articles of manufacture for use with PCR reactions can include nucleotide triphosphates, an appropriate buffer, and a polymerase. An article of manufacture of the invention also can include appropriate reagents for detecting the amplification product. For example, an article of manufacture can include one or more restriction enzymes for distinguishing amplification products from different species of microorganism, or can include fluorophore-labeled oligonucleotide probes for real-time detection of amplification products.

It will be appreciated by those of ordinary skill in the art that different articles of manufacture can be provided to evaluate microbial profiles of different species of animals. For example, the microbial profile of the gastrointestinal tract of a pig has a different community of microbes than that of poultry. Therefore, an article of manufacture designed to evaluate the microbial profile of, for example, a pig may have a different set of controls or a different set of species-specific hybridization probes than that designed, for example, for poultry. Alternatively, a more generalized article of manufacture can be used to evaluate the microbial profile of a number of different animal species.

### Computer System and Software

A computer suitable for use in the invention can be a personal computer owned and operated by an individual or it can be a server or other computer system used by businesses in their operations. For example, a computer can be a server upon which such web pages can be stored and through which the pages are publicly accessible by others. A computer typically includes a main unit that contains the essentials for computing: a processor, a primary memory, a secondary memory, ports and connectors for input and output, and circuitry connecting the components to make the computer functional, as is well known in the art. Output from the computer can be displayed through a monitor that is usually connected to the main unit. Output from the computer can also be transmitted to a printer that is connected to the computer. A user may submit information and commands though various input means as is well known. For example, a user may employ a keyboard and/or a pointing device to enter data and commands. The computer can be connected to a public computer network, such as the Internet, or to a private or semi-private network, through a cable. Other means of connecting to public networks are also contemplated and this description is not limited to any connection in particular. A computer for use in the invention can be a portable device similar to, for example, a personal digital assistant (PDA).

A computer typically operates by executing program steps that may be stored in memory or that may be accessible from sources outside the computer. The computer may access information on a storage medium capable of being read in a media reader. For example, the reader may be capable of reading a computer-readable storage medium such as either or both 3.5″ floppy disks or compact disks (CDs). Accessing sources outside the computer can occur via any suitable means, such as e-mail, hardwire connection, wireless network, or cellular network.

The invention provides for a system that can be used to determine a microbial profile index for one or more animals. A system for determining a microbial profile generally includes means for inputting subject microbial profile information; means for accessing control microbial profile information; and a processor for determining a microbial profile index. A system of the invention can further include means to obtain animal performance information based on the microbial profile index so determined, and also can include means to obtain instructions for improving animal performance.

As used herein, “means for inputting subject microbial profile information” can refer to any method by which microbial profile data is introduced into or obtained by a system of the invention. For example, biological instrumentation can be included in a system of the invention and can be used to obtain and transfer microbial profile data evaluated from a biological sample into the system. Subject microbial profile information also can be directly keyed in using, for example, a keyboard. In addition, subject microbial profile information can be introduced into a system of the invention using a media reader that accesses microbial profile information contained on a floppy disk or CD. Alternatively, subject microbial profile information can be obtained from a remote source such as a database, wherein the system accesses the database via a network connection.

As used herein, “means for accessing control microbial profile information” can include numerous means for acquiring the control microbial profile information necessary to determine a microbial profile index for one or more animals. For example, the same methods described above for inputting subject microbial profile information into a system of the invention can be used to access control microbial profile information. Instrumentation can be used in a system of the invention to evaluate a control animal's microbial profile, or control microbial profile information can be keyed into a system directly. Control microbial profile information also can be provided on a floppy disk or CD for access via a media reader included as part of a system of the invention. Further, a system of the invention can access control microbial profile information remotely (e.g., a remote database) via a network connection. A network connection to access a remote database of control microbial profiles is particularly useful given that a remote-accessed database can be readily updated with control microbial profile information as necessary.

A “processor for determining a microbial profile index” generally refers to a processor that is internal to a system of the invention. Alternatively, a processor for determining a microbial profile index can be a remote processor such as one accessed over a network connection. A remote processor can be, but is not required to be, at the same remote site at which control microbial profile information is accessed.

A system of the invention can further include “means to obtain animal performance information” and may additionally include “means to obtain instructions for improving animal performance.” Generally, the data correlating microbial profile indices with animal performance is obtained from a database. A database can be accessed by a system locally such as from memory or from a floppy disk or CD such as for example, over a network connection. Similarly, instructions for improving animal performance can be obtained locally or remotely via a system of the invention. Obtaining instructions for improving animal performance using a system of the invention may be of primary importance to, for example, a manager of an animal growing or processing operation.

The invention also provides for a computer-readable storage medium. A computer-readable storage medium can be, for example, a floppy disk or a CD. A computer-readable storage medium of the invention contains instructions (e.g., a software application) stored thereon for causing a processor to determine a microbial profile index for one or more animals. The processor determines the microbial profile index by accessing microbial profile information of subject animals and of control animals. In one embodiment, the microbial profile information of subject animals is accessed from memory or from a computer-readable storage medium, while the microbial profile information of control animals is accessed over a network connection. For example, the network connection can access a database of microbial profile information on control animals. A computer-readable storage medium of the invention can further comprise instructions for causing the processor to correlate the microbial profile index with animal performance and outputting performance information. Output of animal performance can be via a monitor or a printer, for example, and can be in any number of formats including, but not limited to, a numerical value that can be correlated with animal performance on a scale of animal performance, or specific instructions regarding one or more feed additives.

The invention will be further described in the following examples, which do not limit the scope of the invention described in the claims.

### EXAMPLES

### Example 1

### Animals

Twenty-four pigs were weaned at 19 days of age and allotted in a completely randomized block design to one of two treatments: a corn/soy diet or a corn/soy diet supplemented with 0.75% sodium benzoate. A two-phase feeding program was employed with both diets being formulated to meet the dietary recommendations for pigs of this age.

All pigs were weighed at days 0, 12, and 27, and feed intake was recorded daily. Performance data were analyzed as a randomized complete block design and differences between treatments were compared using Student's t-test.

### Example 2

### Sampling of Intestinal Digesta

On days 13 and 27, pigs were sacrificed by means of a captive bullet, and the gastrointestinal tract (GIT) from each was excised. Digesta from the lumen of four regions of the GIT—pyloric region of the stomach, duodenum/jejunum junction, mid-ileum, and mid-cecum—was carefully removed for microbiological analysis. All samples were collected with minimal exposure to oxygen, purged with nitrogen gas, and transported to the laboratory on ice. Within 24 hr of collection, the samples were enumerated for select bacterial populations (total anaerobic heterotrophs, lactobacilli, and enterics (e.g., Escherichia coli, Salmonella spp., etc.) using standard plating techniques and then stored at—80° C. for subsequent molecular analysis.

### Example 3

### Analysis of Intestinal Digesta for Microbiology

Cultivation-Based Analysis: At analysis, individual digesta samples were diluted 10-fold in sterile anaerobic buffer. To determine the total heterotrophic anaerobe population in each sample, as well as to estimate of the level of each bacterial type to be enumerated, a microtiter plate-based most probable number (MPN) assay was employed. For this, 100 μl of a 1:10 dilution of each digesta sample was inoculated in triplicate into MPN microtiter plates containing reinforced rumen clostridial medium (RCM). The inoculated MPN plates were incubated in an anaerobic chamber at 39° C., for up to 72 hours. Following incubation, a transilluminant light box was used to check for growth in each dilution well by comparing the turbidity to uninoculated control wells. The number of triplicate wells that showed growth was recorded, and three-tube MPN tables were used to determine the MPN values (see, for example, Coch, Growth Measurement, Ch 11, In: Manual of Methods for General Bacteriology, Gerhardt, ed., American Society for Microbiology, Washington D.C., 1981).

For viable counts of lactobacilli, the appropriate serial dilutions of digesta were plated on LAMVAB (Lactobacillus anaerobic MRS with vancomycin and bromocresol green) agar and incubated anaerobically. Enterobacteriaciae were enumerated using MacConkey's agar with aerobic incubation. All plates were incubated at 39° C., for 72 h. Following incubation, dilutions that gave rise to 30-300 colonies per plate were counted using a colony counter. Analysis of variance (ANOVA) and Tukey (p≦0.15) were used to determine microbial differences due to treatment.

Molecular analyses: Total genomic DNA from the digesta was extracted using a bead-beating method followed by QiaAmp column purification. The DNA was quantified by absorbance at 260 nm and stored at −20° C. The V3 region of the 16S rRNA gene was amplified by PCR using two universal primers for Eubacteria. The forward primer had a 40-base GC clamp attached to its 5′ end. Aliquots (100 ng) of genomic DNA were used in each PCR reaction. Following each PCR, the products were analyzed by electrophoresis on 1.5% (w/v) agarose gels to check for the presence of the expected PCR products. The expected PCR products were about 200 bp in size. When necessary, a second PCR reaction was repeated with 200 ng genomic DNA. The PCR products were separated at 60° C. on DGGE gels with a denaturant gradient of 40-56%. After staining with GelStar, the banding patterns were recorded using an AlphaImager.

### Example 4

### Performance

Weanling pigs were used to determine if supplementing diets with sodium benzoate would beneficially modulate the GIT microflora and result in improved growth performance. Early weaning occurs at a time when the anatomical, physiological, and microbiological characteristics of the piglet's digestive system are still immature. As a result, weaning places considerable nutritional and physiological stress on the animal, often causing digestive upsets which are reflected in poor health and performance. Well-managed animals surviving these stresses go on to develop a stable digestive system—the microbial ecosystem thus being less susceptible to intervention by means of diet.

Here, piglets were weaned at 19 days of age to promote an imbalance in the GIT ecosystem. Growth performance measurements of pigs over the course of this study are summarized in Table 1. The addition of sodium benzoate to the diet significantly increased average daily gain (ADG) and average daily feed intake (ADFI).

TABLE 1
Growth performance of weanling pigs fed a corn/soy diet with
and without sodium benzoate

Treatment

| Parameter | Control (basal) | Sodium Benzoate |
|---|---|---|
| Phase 1 | | |
| Gain* (lbs/day) | 0.32 | 0.44 |
| Feed intake* (lbs/day) | 0.43 | 0.54 |
| Feed:Gain | 1.41 | 1.26 |
| Phase 2 | | |
| Gain** (lbs/day) | 0.86 | 1.00 |
| Feed intake (lbs/day) | 1.27 | 1.38 |
| Feed:Gain** | 1.48 | 1.40 |

*statistically significant at P ≦ 0.05
**statistically significant at P ≦ 0.20

During the second period (15 days in length), sodium benzoate-fed pigs had improved gain and conversion (p=0.18 and p=0.20, respectively), and had a numeric improvement in ADFI (Table 1). In addition, feeding sodium benzoate resulted in a significant reduction in GIT pH as compared to pigs fed the control diet. As measured on day 13, sodium benzoate-fed pigs had an ileal pH of 5.90 (p=0.04) and a cecal pH of 5.54 (p=0.22), compared to pHs of 6.51 and 5.80 in the ileum and cecum, respectively, of pigs fed the control diet. By day 28, gastrointestinal pH was consistent across both treatments. There was a significant increase in protein digestibility (p<0.05) at the ileum for pigs fed sodium benzoate versus the negative control.

### Example 5

### Microbiology

Digesta samples were analyzed for total anaerobic bacteria, total lactobacilli, and total enterics (e.g., E. coli & Salmonella spp.). Microbiological analysis of digesta from four regions along the GIT of treated pigs at 13- and 27-days post-weaning suggested that sodium benzoate affected the microbial ecosystem.

Total Anaerobes: Table 2 summarizes the total anaerobic counts obtained at both sampling periods. At 13-days post-weaning, as might be expected, higher mean levels of total anaerobes were obtained from digesta samples collected from the ileum (6.32±0.92 log₁₀ MPN/g) and cecum (7.95±0.39 log₁₀ MPN/g) than from the stomach (5.70±1.2 log₁₀ MPN/g) or the duodenum (5.25±0.82 log₁₀ MPN/g). Compared to the negative control, sodium benzoate numerically increased the counts from digesta obtained from the ileum and cecum, but had little effect on the anaerobic heterotrophs higher up in the GIT. By day 27, total anaerobic heterotrophs increased in all regions of the GIT sampled independent of treatment (Table 2). Between the two periods, the counts obtained from cecal, duodenal and stomach digest a appear to have “stabilized,” and by the methods employed here, there were no clear differences between the treatment groups.

TABLE 2
Total anaerobic bacteria at 13- and 27-days post-weaning in
the GIT of pigs treated with and without sodium benzoate

| | | | Counts | |
|---|---|---|---|---|
| | | Intestinal | (Log₁₀ | |
| Treatment | Day | Segment | MPN/g) | CV (%) |
| Control (basal) | 13 | Stomach | 4.61 | 4.91 |
| | | Duodenum | 4.56 | 0.00 |
| | | Ileum | 5.75 | 23.27 |
| | | Cecum | 7.97 | 0.00 |
| | 27 | Stomach | 5.45 | 21.25 |
| | | Duodenum | 6.11 | 13.15 |
| | | Ileum | 7.70 | 17.39 |
| | | Cecum | 9.34 | 9.67 |
| Sodium | 13 | Stomach | 4.50 | 1.02 |
| benzoate | | Duodenum | 4.67 | 5.60 |
| | | Ileum | 6.60 | 24.25 |
| | | Cecum | 7.76 | 22.82 |
| | 27 | Stomach | 6.03 | 15.2 |
| | | Duodenum | 6.60 | 8.36 |
| | | Ileum | 6.28 | 27.86 |
| | | Cecum | 9.38 | 0.00 |

lactobacilli: At day 13, mean lactobacilli counts in the negative control treatment range from 2.19 to 8.39 log ₁₀ CFU/g in the duodenum/jejunum and cecum, respectively (Table 3). Sodium benzoate appeared to stimulate lactobacilli in the ileum of weanling pigs. The magnitude of lactobacilli in the stomach—6.39 log₁₀ CFU/g at day 13 and 5.62 log₁₀ CFU/g at day 27—as determined by plating on LAMVAB medium was rather surprising. This medium is nutrient rich and acidified (pH 5.0) with vancomycin. It is possible that bacteria cultured on these plates were not lactobacilli but rather acid tolerant, vancomycin resistant bacteria. By day 27, similar to the anaerobe counts, there was little difference between the treatments suggesting that the lactobacilli population in the GIT had matured and stabilized.

TABLE 3
Total lactobacilli* in the GIT of pigs at 13- and 27-days
post-weaning treated with and without sodium benzoate

| | | Intestinal | Counts | |
|---|---|---|---|---|
| Treatment | Day | Segment | (Log₁₀ CFU/g) | CV (%) |
| Control (basal) | 13 | Stomach | 7.53 | 13.96 |
| | | Duodenum | 2.19 | 173.2 |
| | | Ileum | 6.78 | 5.83 |
| | | Cecum | 8.39 | 8.64 |
| | 27 | Stomach | 5.77 | 23.15 |
| | | Duodenum | 6.01 | 2.54 |
| | | Ileum | 4.79 | 90.34 |
| | | Cecum | 9.11 | 2.31 |
| Sodium benzoate | 13 | Stomach | 7.53 | 13.96 |
| | | Duodenum | 3.77 | 86.61 |
| | | Ileum | 8.08 | 1.01 |
| | | Cecum | 8.46 | 4.97 |
| | 27 | Stomach | 6.49 | 5.39 |
| | | Duodenum | 7.09 | 6.13 |
| | | Ileum | 4.77 | 86.60 |
| | | Cecum | 9.11 | 2.31 |

*Lactobacilli as determined by LAMVAB medium.

Enteric Bacteria: At day 13, the culturable counts on MacConkey agar were less than 10⁴ CFU/g for most samples other than cecal digesta. Measurable counts were also obtained from ileal digesta collected from the sodium benzoate treatment groups (Table 4). By day 27, mean enteric counts greatly increased in the duodenal and ileal digesta for both treatment groups. Overall, there appeared to be limited effects on the enteric populations attributable to the sodium benzoate feed additive.

TABLE 4
Total enteric bacteria in the GIT of pigs at 13- and 27-days
post-weaning treated with and without sodium benzoate

| | | Intestinal | Counts | |
|---|---|---|---|---|
| Treatment | Day | Segment | (Log₁₀ CFU/g) | CV (%) |
| Control (basal) | 13 | Stomach | 0.00 | 0.00 |
| | | Duodenum | 0.00 | 0.00 |
| | | Ileum | 0.00 | 0.00 |
| | | Cecum | 4.17 | 86.71 |
| | 27 | Stomach | 1.23 | 173.21 |
| | | Duodenum | 5.89 | 29.37 |
| | | Ileum | 4.36 | 91.80 |
| | | Cecum | 6.81 | 16.52 |
| Sodium benzoate | 13 | Stomach | 0.00 | 0.00 |
| | | Duodenum | 0.00 | 0.00 |
| | | Ileum | 3.88 | 86.79 |
| | | Cecum | 5.81 | 9.94 |
| | 27 | Stomach | 1.00 | 173.21 |
| | | Duodenum | 4.29 | 20.32 |
| | | Ileum | 5.89 | 5.92 |
| | | Cecum | 6.14 | 16.13 |

### Example 6

### Molecular Ecology

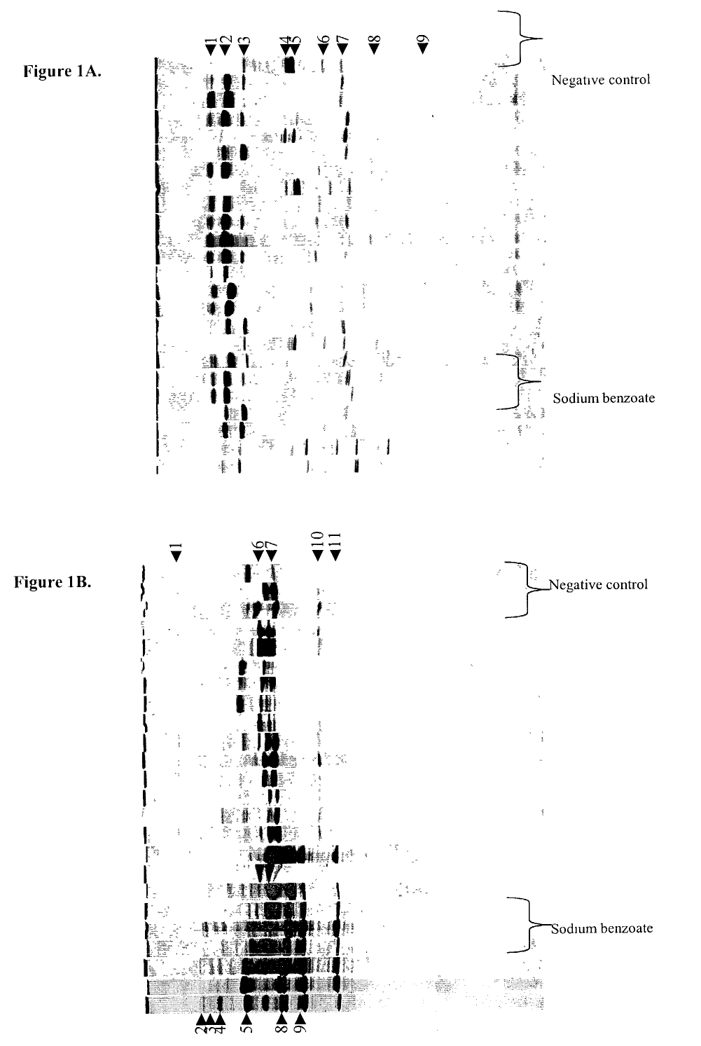

A comparison of 16S rRNA sequences retrieved from intestinal contents was performed. For example, the banding pattern of stomach digesta samples collected on day 13 showed one or two intense bands, suggesting the existence of one or two predominant populations. As indicated by the appearance of bands in the lower half of the gel and overall good separation of the bands, the microbial community in the stomach digesta has a high population density (including high G+C organisms) (FIG. 1A). At day 27, the microbial community in the stomach digesta appeared to be influenced by the sodium benzoate treatment as evidenced by the large increase in the number of bands (FIG. 1B). It is noteworthy that this increase in microbial diversity was not readily observed by the cultivation-based methods.

This study revealed that sodium benzoate significantly improved ADG and ADFI in pigs over the first 14 days post-weaning, and in the subsequent 15 days, significantly improved gain as compared to the pigs fed the basal diet devoid of the organic acid. The intestinal microbial ecosystem is dynamic and is easily altered by, for example, the animal's diet. As determined in this study, at day 13, sodium benzoate-fed pigs harbored more anaerobic heterotrophs and lactobacilli in the mid- to lower GIT than did pigs fed the basal diet. Moreover, the pH in these regions of the GIT was significantly lower than in the control pigs, suggesting that the increased concentrations of lactic acid bacteria were metabolically active. Fewer E. Coli and other coliforms might be expected in the intestinal ecosystem since the increased number of lactobacilli likely resulted in a reduction of intestinal pH through production of lactic acid, but such was not the case. By day 27, it appears that while the diversity of the microbial community had increased, it had also matured and stabilized such that sodium benzoate was less able to affect the microbial profile. A genetic fingerprint of the stomach ecosystem revealed an increase in the microbial diversity between the two sampling periods, and revealed differences in the profiles of sodium benzoate-fed pigs as compared to pigs fed the basal control diet. Taken together, these data suggest that modulation of the GIT microflora can affect animal performance.

### Example 7

### Birds

At 1 day of age, four hundred broilers were randomly allotted to two treatment groups, and housed in mini-floor pens for the duration of the 7-week study. The birds were fed a four-phase corn/soy diet supplemented with or without a blend of organic acids.

At days 1, 16, 32, 42, and 50, all birds were weighed and feed intake was recorded. Performance data were analyzed as a randomized complete block design and differences between treatments were compared using analysis of variance and Tukey's mean separation.

### Example 8

### Microbiology

At day 50 (end of study), intestinal digesta was collected from the cecal tonsil of 18 broilers from each treatment group to assess pathogen load. The pathogens that were initially tested for included Salmonella spp., Campylobacter spp., and Escherichia coli.Within each treatment, digesta from three birds per pen was pooled. At analysis, a sub-sample of each digesta sample was blended, diluted in phosphate buffer, and Spiral Plated on brilliant green agar, Campylobacter agar supplemented with Preston's supplement, and MacConkey agar, respectively for the three test bacteria. Following incubation, colonies were counted using standard Spiral Plating techniques.

### Example 9

### Performance

Broilers were used to determine if a diet supplemented with a blend of organic acids would beneficially modulate the gastrointestinal microflora. Growth (e.g., body weight, and feed conversion) were used a measures of animal performance.

Body Weight—As shown in Table 5, no significant difference was detected in broiler body weight at day 16 (end of the Starter Phase). Consistently throughout the weigh points, birds fed the diet supplemented with the acid blend exhibited a lighter body weight as compared to birds fed the diet without the acid blend. In general, the birds fed the diet supplemented with the blend of organic acids tended to have poor uniformity across replicates. This result correlated with this treatment group generally having a poorer body weight performance.

TABLE 5
Effect of a diet supplemented with an organic acid blend on
body weight and uniformity of broilers

| Treatment | | Day 16 | Day 32 | Day 42 | Day 50 |
|---|---|---|---|---|---|
| Control | kg | 0.337 | 1.342 | 2.082 | 2.643 |
| (Basal) | SD | 0.02 | 0.058 | 0.115 | 0.185 |
| | CV | 6.32 | 4.32 | 5.52 | 6.98 |
| Acid Blend | kg | 0.343 | 1.333 | 2.037 | 2.548 |
| | SD | 0.020 | 0.029 | 0.159 | 0.188 |
| | CV | 5.94 | 2.17 | 7.82 | 7.39 |

kg = kilogram; SD = standard deviation; CV = coefficient of variance

Feed Conversion—The results of feed conversion experiments are shown in Table 6. Although no statistical differences were detected, birds fed the diet supplemented with the acid blend had a poorer numeric feed conversion throughout the study than did birds fed a control diet. Similarly, when corrected for mortality and for mortality (+) body weight, the diet supplemented with the acid blend negatively affected feed conversion.

TABLE 6
Effect of a diet supplemented with an acid blend
on the feed conversion of broilers

| | | | | |
|---|---|---|---|---|
| Treatment | Day 16 | Day 32 | Day 42 | Day 50 |

Unadjusted Feed Conversion
(kg feed/kg body weight gain)

| | | | | |
|---|---|---|---|---|
| Negative Control | 1.342 | 1.594 | 1.737 | 1.947 |
| Acid Blend | 1.344 | 1.600 | 1.762 | 1.967 |

Mortality Corrected Feed Conversion (kg
feed/kg body weight gain)

| | | | | |
|---|---|---|---|---|
| Negative Control | 1.307 | 1.594 | 1.730 | 1.894 |
| Acid Blend | 1.309 | 1.600 | 1.766 | 1.913 |

Body Weight (+) Mortality Corrected
Feed Conversion
(kg feed/kg body weight gain)

| | | | | |
|---|---|---|---|---|
| Negative Control | 1.307 | 1.583 | 1.724 | 1.894 |
| Acid Blend | 1.307 | 1.590 | 1.760 | 1.956 |

### Example 10

### Microbiology

Results of microbial analyses of the cecal contents of 50-day old broilers for common poultry pathogens are presented in Table 7. The total number of coliforms (as represented by E. coli) was not significantly different (p=0.15) in birds fed the control diet and birds fed the diet supplemented with the acid blend—approximately 8.0 log₁₀ CFU/g in all birds sampled.

Interestingly, the birds fed the diet supplemented with the acid blend harbored a slightly reduced population of Campylobacter spp. than birds fed the diet without the acid blend. Salmonella was not detected in the cecal contents of any of the birds tested, indicative of the clean conditions in the research facility where the trial was conducted.

TABLE 7
Microbial analysis of cecal digesta contents from 50-day old broilers

| Dietary | Log₁₀ CFU/g digesta | | |
|---|---|---|---|
| Treatment | E. coli | Salmonella | Campylobacter |
| Negative control | 8.00 | ND* | 2.63 |
| Acid Blend | 7.90 | ND* | 1.78 |

*ND = not detected

The results of this study show that supplementing a corn/soy broiler diet with a blend of organic acids reduced the microbial load of at least one common poultry pathogen as compared to birds fed a diet without the acid blend. Birds fed the diet supplemented with the acid blend also showed poorer performance than birds fed a control diet as determined by body weight, uniformity, and feed conversion. Although not bound by any particular theory, this phenomenon may be attributable to, for example, a concomitant decrease in the microbial load of a beneficial microbe or a concomitant increase in the microbial load of a different (i.e., non-Campylobacter) pathogenic microbe.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of characterizing animal performance, comprising:
determining a microbial profile in a biological sample from one or more animals;
comparing said profile with a control microbial profile to determine a microbial profile index; and
correlating said index with said animal performance.

2. The method of claim 1, wherein said characterizing is performed on a plurality of animals.

3. The method of claim 1, wherein said animal is avian.

4. The method of claim 3, wherein said avian animal is a poultry animal.

5. The method of claim 1, wherein said is porcine.

6. The method of claim 1, wherein said animal is ruminant.

7. The method of claim 1, wherein said animal is a companion animal.

8. The method of claim 1, wherein said animal is aquatic.

9. The method of claim 1, wherein said animal is human.

10. The method of claim 1, wherein said animal performance is growth.

11. The method of claim 1, wherein said animal performance is weight gain.

12. The method of claim 1, wherein said animal performance is lean body mass.

13. The method of claim 1, wherein said animal performance is pigmentation.

14. The method of claim 1, wherein said animal performance is intake feed conversion.

15. The method of claim 1, wherein said animal performance is mortality.

16. The method of claim 1, wherein said animal performance is morbidity.

17. The method of claim 1, wherein said animal performance is rate of condemnation.

18. The method of claim 1, wherein said animal performance is happiness.

19. The method of claim 1, wherein said animal performance is health.

20. The method of claim 1, wherein said animal performance is lack of sickness/illness.

21. The method of claim 1, wherein said determining comprises microbial culturing, and colony identification.

22. The method of claim 21, wherein said determining further comprises enumerating said colonies.

23. The method of claim 1, wherein said determining comprises histological analysis.

24. The method of claim 1, wherein said determining comprises immunological analysis.

25. The method of claim 1, wherein said determining comprises genetic fingerprinting.

26. The method of claim 1, wherein said determining comprises 16S rDNA genotyping.

27. The method of claim 1, wherein said determining comprises cpn60 genotyping.

28. The method of claim 1, wherein said control microbial profile is from a control biological sample taken from said animal prior to or subsequent to the taking of said biological sample.

29. The method of claim 1, wherein said control microbial profile is from a control biological sample taken from another animal.

30. The method of claim 1, wherein said control microbial profile is from control biological samples taken from a plurality of animals.

31. The method of claim 30, wherein said biological samples are pooled.

32. The method of claim 1, wherein said baseline microbial profile is from a database of microbial profiles.

33. The method of claim 1, wherein said biological sample is a gastrointestinal tract sample.

34. The method of claim 33, wherein said gastrointestinal tract sample is digesta.

35. The method of claim 33, wherein said gastrointestinal tract sample is mucous and/or mucosal tissue.

36. The method of claim 33, wherein said gastrointestinal tract sample is feces.

37. The method of claim 1, wherein said biological sample is a periodontal sample.

38. The method of claim 1, wherein said biological sample is bodily fluid.

39. The method of claim 38, wherein said bodily fluid is selected from the group consisting of blood, urine, saliva, sputum, or semen.

40. The method of claim 1, wherein said biological sample is a soil sample.

41. The method of claim 1, wherein said biological sample is an aqueous sample.

42. The method of claim 1, wherein said microbial profile is based on taxonomic and phylogenetic identification of microbes in said biological sample.

43. The method of claim 42, wherein said microbes are selected from the group consisting of bacteria, protozoa, and fungi.

44. A method of monitoring the effect of a feed or feed additive regimen on the microbial profile of one or more animals, comprising:
providing said feed or feed additive regimen to said one or more animals;
determining a post-regimen microbial profile of said one or more animals; and
comparing said post-regimen microbial profile with a pre-regimen microbial profile of said one or more animals.

45. The method of claim 44, further comprising:
adjusting said feed or feed additive regimen based upon said comparison; and
determining an adjusted-regimen microbial profile for said one or more animals.

46. The method of claim 45, further comprising:
correlating said pre-regimen microbial profile, said post-regimen microbial profile, and/or said adjusted-regimen microbial profile with animal performance.

47. The method of claim 44, wherein said feed additive comprises one or more components selected from the group consisting of medicated feed additives, enzymes, probiotic microbials, direct-fed microbials, antimicrobials, prebiotics, phytochemicals, immunomodulators, antibodies, plant extracts, essential oils, organic acids, antioxidants, amino acids, oligosaccharides, oleoresins, herbs, spices, saponins, and sea plants.

48. An article of manufacture, comprising:
at least one cpn60 oligonucleotide; and
instructions therein for using said cpn60 oligonucleotide(s) to characterize animal performance or to monitor the effects of a feed or feed additive on the microbial profile of said animal.

49. The article of manufacture of claim 48, wherein said cpn60 oligonucleotide(s) are attached to a microarray.

50. A computer-readable storage medium having instructions stored thereon for causing a programmable processor to:
determine a microbial profile index for one or more animals based upon a microbial profile of said one or more animals.

51. The computer-readable storage medium of claim 49, further comprising instructions for causing said programmable processor to:
correlate said microbial profile index with a performance characteristic of said animal.

52. The computer-readable storage medium of claim 49, further comprising instructions for causing said programmable processor to:
compare two profiles, wherein said comparison determines discreet differences between said profiles.

53. A system for determining a microbial profile index for one or more animals, the system comprising:
means for inputting subject microbial profile information;
means for accessing control microbial profile information; and
a processor for determining a microbial profile index.

54. The system of claim 53, further comprising:
means to obtain animal performance information based on said microbial profile index.

55. The system of claim 53, further comprising:
means to obtain instructions for improving said animal performance.

56. A method of managing an animal growing operation or an animal processing operation, comprising:
providing a microbial profile index for one or more animals; and
communicating said index to a manager of said animal growing operation or said animal processing operation.

57. The method of claim 56, further comprising:
correlating said index with animal performance.

58. The method of claim 56, further comprising:
adjusting a feed or feed additive regimen.

59. The method of claim 56, further comprising:
modifying said animal processing operation.
